(19) United States
(12) Patent Application Publication
(10) Pub. No.: US 2009/0292193 A1
(43) Pub. Date: Nov. 26, 2009

(54) ELECTRODES OR SENSORS ENCAPSULATING EMBODIMENT FOR WEARABLE PHYSIOLOGICAL INFORMATION MONITORING STRAPS AND GARMENTS AND THEIR CONSTRUCTION METHODS

(76) Inventor: RAVINDRA WIJESIRIWARDANA, Bentonville, AR (US)

Correspondence Address: RAVINDRA WIJESIRIWARDANA, 2001 LATROBE DRIVE APT 20, BENTONVILLE, AR, 72712, US

(21) Appl. No.: 12/401,789

(22) Filed: Mar. 11, 2009

Related U.S. Application Data
(60) Provisional application No. 61/035,852, filed on Mar. 12, 2008.

(51) Int. Cl. A61B 5/04; A61M 31/00

(52) U.S. Cl. 600/388; 604/48

(57) ABSTRACT

The present invention describes physiological sensors embodiment having a pressure ring around the sensors. The pressure ring embodiment is compressible and stretchable and non shearable. One or more electrode sensors embodiments are integrated to a stretchable strap that can be worn by a person on the torso. This system is capable of monitoring and measuring physiological information of the wearer more accurately and reliably and providing a comfortable to wear platform at the same time. Further the sensors in the embodiment can be replaced by actuators and used as an actuator embodiment with lesser motion artifacts.

### CROSS-REFERENCE TO RELATED APPLICATIONS

This application claims the benefit of provisional patent application Ser. No. 61/035,852, filed 2008 Mar. 12 by the present inventor.

### FEDERALLY SPONSORED RESEARCH

Not Applicable

### SEQUENCE LISTING OR PROGRAM

Not Applicable

### BACKGROUND OF THE INVENTION

1. Field

This application relates to bio-potential electrodes and sensors based wearable physiological information monitoring straps and garments.

2. Prior Art

Wearable physiological information systems are made by integrating a physiological sensor into the wearable devices including straps, garments and wrist worn head worn devices. Even though these systems have more than 100 years of history, one common problem affects the performance of all these systems. That is these systems fail to perform under most demanding situations such as when a wearer's body undergoes motion

Several approaches have been made to overcome this in wearable electrodes based straps and garments. One approach discussed in U.S. Pat. No. 6,553,247 introduces electrodes with a wave profile so that the electrode will connect to the body with a pressure contacts. However this approach is limited since it exerts high pressure points on the skin and also when a person sweats the electrode slips and hence makes it unreliable in most applications. Another approach in U.S. Pat. No. 6,580,943 describes the application of backing material pads between the electrodes and the straps so that the pressure can be applied on to the skin to keep the electrode stable. In this approach again it is very uncomfortable to wear and also when the motion level increases the noise level increases and the accuracy of the readings is lowered. Many variants of these two methods can be found in the literature. Most of these systems are based on fabric straps and garments and use for measuring heart rate with electrodes as the sensors.

The present invention introduces a new encapsulating unit that consists of a ring around the sensor or the electrode so that it holds the electrode in place not allowing the electrode to move against the skin. Therefore the above limitations can be overcome in the electrodes or sensors based physiological information monitoring garments and straps.

The first part of the invention discusses an embodiment that can be used to incorporate a physiological information monitoring sensor such as an electrode, temperature sensor or a pressure and displacement transducer, optical transducer (pulse plethyismography (PPG)) hydration sensor. The embodiment is shown in the FIG. 1A. It consists of an outer ring that pressed against the body surface (electrically non conductive materials) of the wearer. This ring is connected to the rest of the embodiment or extends from the same embodiment that connects to the stretchable substrate. This arrangement can be fabricated by using layers approach or by extending the same materials of the substrate using any fabricating method. The ring surface that pressed against the body may contain micro or nano scale spikes, adhesive layer, any surface morphological texture that improves the adhesion or the contact to the skin of the wearer. The ring material may be compressible as well.

Materials of the embodiment consists of stiff body that would compress (006 direction) or bends (005 directions) and has very little shear in the directions 010 and 011. This is to make the inner surface of the embodiment or the sensor area that holds the electrodes or the sensors contacted stably with the skin of the wearer. This is achieved by using pressure on the two surfaces (sensors and the skin) by the external stretchable strap or garment.

### DRAWINGS—FIGURES

FIG. 1A—The electrodes or sensors embodiment integrated into the stretchable substrate.

FIG. 1B—Two electrodes or sensors embodiments integrated into the stretchable substrate.

FIG. 2A—TOP VIEW of the electrodes or sensors embodiment.

FIG. 2B—SIDE VIEW of the electrodes or sensors embodiment.

FIG. 2C—CROSS SECTIONAL VIEW of the electrodes or sensors embodiment that shows the skin contacts of the electrodes, sensors and the ring.

FIG. 3A—ECG signal picked up from two electrode embodiments based strap running at 9-10 mph running and sweaty condition

FIG. 3B—ECG signal picked up from a market leading electrodes strap running at 9-10 mph running and sweaty condition

FIG. 3C—Heart rate picked up from two electrode embodiments based strap running at 9-10 mph running and sweaty condition

FIG. 3D—Heart rate picked up from a market leading electrodes strap running at 9-10 mph running and sweaty condition

### DRAWINGS—REFERENCE NUMERALS

001—Outer ring of the electrodes or sensors embodiment.
002—Stretchable substrate.
003—Electrodes or Sensors
004—Conductive pathways that carries the power signal lines from and to of the sensors or the electrodes in the embodiment.
005—Center axis
010—X axis direction
011—Y axis direction
006—Z axis direction or the direction of compress
007—Skin surface of a wearer
008—Sensor mount layer of the electrodes or sensors embodiment
009—The body of a wearer

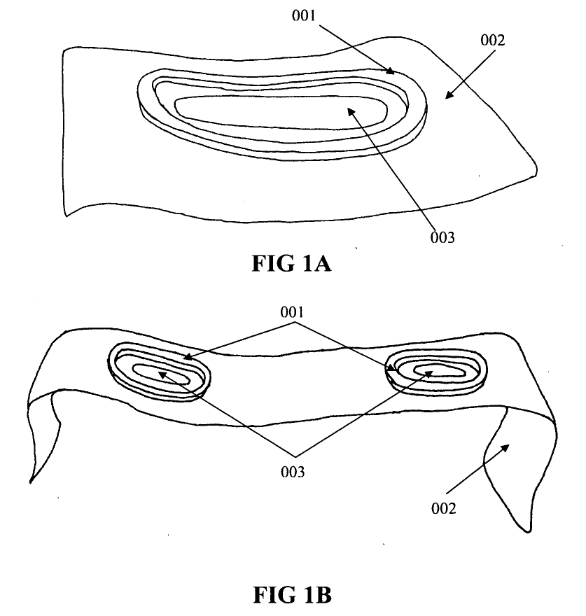

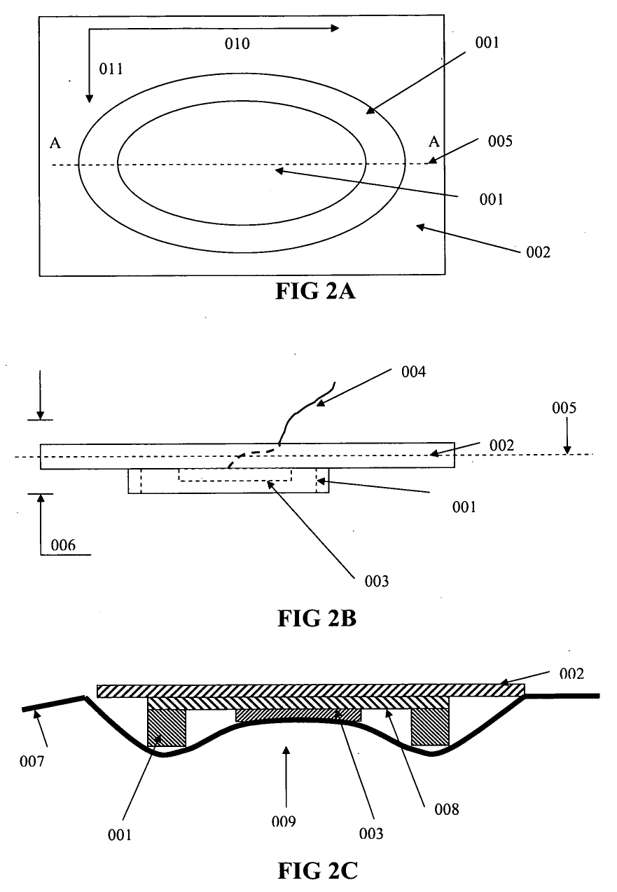

### DETAILED DESCRIPTION OF FIG. 1A, FIG. 1B, FIG. 2A, FIG. 2B, FIG. 2C

FIG. 1A shows the electrodes or the sensors embodiment connected to the stretchable substrate (002). The embodiment consists of and outer ring (001) and sensors or electrodes in the inside surface of the embodiment, so that they can touch and press against the skin of a wearer upon wearing. The embodiment can be attached to the stretchable substrate.

FIG. 1B shows two electrodes or sensors embodiments integrated in to a stretchable wearable substrate. There can be more than two electrodes or sensors embodiments on the substrate depending on the application.

FIG. 2A shows the TOP VIEW of the electrodes or sensors embodiment. The ring (001) in this figure has an oval shape. However this shape could be a circle, a square or a rectangle.

FIG. 2B shows the SIDE VIEW of the electrodes or sensors embodiment. That sows the conduction pathways (004) and the Z direction (006). The whole embodiment is compressible in the Z direction and stretchable in the X direction. However it is not stretchable in the X direction.

FIG. 2C shows the CROSS SECTIONAL VIEW of the electrodes or sensors embodiment. This shows the how the ring is pressed on to the skin surface of the wearer. Also shows how the sensors or the electrode are touching the skin.

### OPERATION OF THE INVENTION

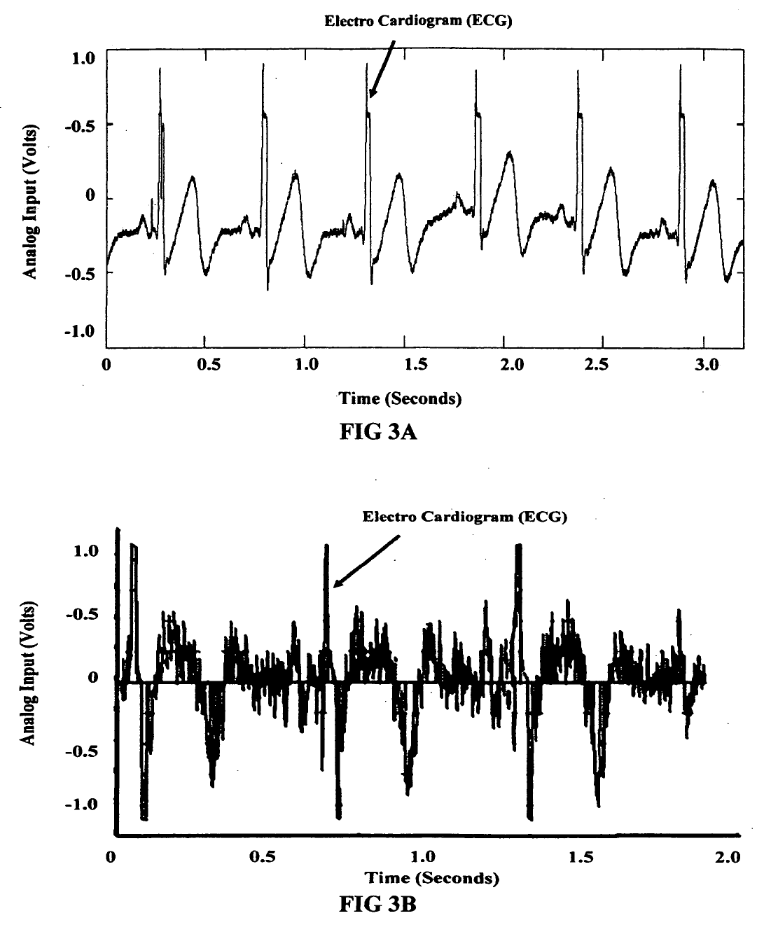

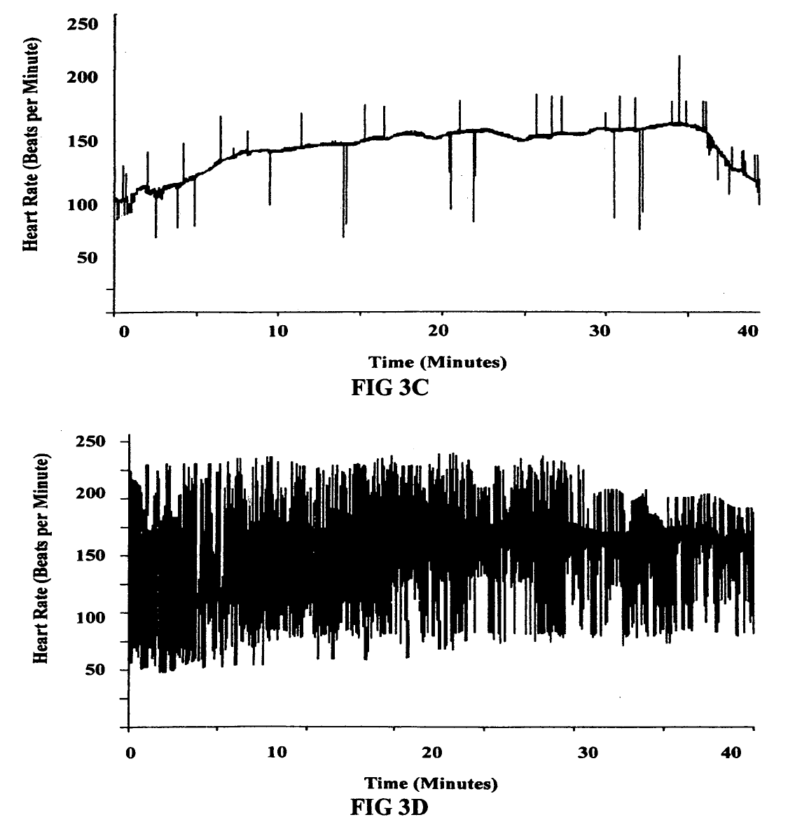

The sensors or the electrodes of the embodiment are in contact with the skin. The outer ring of the embodiment presses against the skin of the wearer and hence holds the embodiment stationary against the skin. This is achieved by the pressure between the contact surfaces. The embodiment is on the stretchable substrate strap. When a person wears this strap it stretches and this tensile force is transferred to the surface of the ring and the senor surface makes the surfaces to press against the skin. During motion the ring stops the vibrations to move into the sensor area and hence reduce the motion artifacts effect. In addition this arrangement makes the requirement of the applied pressure in order to maintain the contact is minimized and hence providing the comfort for the wearer. When a sensor unit capable of attaching to skin is used the requirement of the pressure is further reduced. The system is tested against the market leading fabric electrodes straps and hard straps. The results are shown in the FIG. 3A, FIG. 3B, FIG. 3C and FIG. 3D. FIG. 3A shows the ECG signal picked up from two electrode embodiments based (made according to the present invention) strap at 9-10 mph running under sweaty condition. FIG. 3B shows an ECG signal picked up from the same person wearing a market leading electrodes strap and running under the same conditions. FIG. 3C shows the heart rate picked from two electrode embodiments based strap at 9-10 mph running under sweaty condition. FIG. 3D shows the heart rate picked up from the same person wearing a market leading electrodes strap and running under the same conditions. The percentage heart rate error is calculated by dividing the number of error readings from the total number of heart rate readings. 10-15% of error rate is observed with the present invention and 80-90% of error rate is observed with market leading monitors under same conditions. The present invention is capable of minimum 4 to 5 times better performance under high motion and sweaty conditions. It is clear from these results that the present invention is a better performing system having better accuracy, reliability and comfort level to wear.

I claim:

1. A sensor embodiment or encapsulating device that can be connected embedded on or in a stretchable material worn by a mammal that comprises of at least one an electrically insulating ring that compresses the skin of the wearer so that the skin inside the ring lifts up and touches with the sensor elements of the embodiment such that.
(a) The sensors are connected or embedded the inside surface of the embodiment encircled by the ring.

2. A device according to claim 1 where the sensors are an bio potential electrodes, pulse oximetry or pulse plethysmography (PPG/SpO₂) sensors, thermal sensors, blood gas sensors or chemical sensors.

3. An embodiment according to claim 1 having actuators instead of the sensors inside surface of the embodiment that touches the skin of the wearer.

4. The actuators in claim 4 comprise of heaters, electrodes or trans epidermal drug delivery units.

5. Use of one or more sensor embodiments in claim 1 or measuring or monitoring at least one physiological signal characteristics or event of the wearer.

6. A device according to claim 1 where the embodiment sensors are electrically connected with a signal or power caring cables

7. The embodiment according to claim 1 made out of materials that are allowed to compress in the direction 006, bendable in the direction 005 and not stretchable or not shearable along the directions 010 and 011.

8. A device according to claim 1 where the construction can be carried out via mechanically chemically or thermally connecting layers or by using a mould.

9. A device according to claim 1 where the materials used in the embodiment are being foam, rubber, plastic, polymeric, ceramic and any combination of any of these.

10. A device according to claim 1 where the sensor is embedded or integrated together with the stretchable material and then the embodiment is put in either by using molding or layer construction methods or combination of both approaches.

11. A device according to claim 1 where the device can be made by using flexible materials such as polymeric materials and a stiffener is used to get the required stiffness and mechanical properties of the embodiment.

12. The sensor surface that touches the skin according to a device in claim 1 having an electro conductive or thermal conductive adhesive layer.
